(19) United States
(12) Patent Application Publication
(10) Pub. No.: US 2009/0326597 A1
(43) Pub. Date: Dec. 31, 2009

(54) SOLAR CELL FOR IMPLANTABLE MEDICAL DEVICE

(75) Inventor: Nathan Zommer, Fort Lauderdale, FL (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: IXYS Corporation, Milpitas, CA (US)

(21) Appl. No.: 12/490,198

(22) Filed: Jun. 23, 2009

Related U.S. Application Data
(60) Provisional application No. 61/075,945, filed on Jun. 26, 2008.

(51) Int. Cl. A61N 1/378

(52) U.S. Cl. 607/5; 607/33; 607/61

(57) ABSTRACT

An implantable medical device includes a solar cell configured to provide energy to recharge a power source such as a battery. The power source is coupled to a control circuit of the medical device and provides power to the circuit. The solar cell may be coupled to the power source via a wire and may be distanced from a housing of the medical device. The solar cell may also be attached to the housing or may be disposed in the housing. The medical device may be implanted in the body of a host such that a surface of the solar cell is provided under a layer of skin of the host. The translucent property of skin allows the solar cell to receive light or infrared radiation from outside the body. The solar cell converts the received energy and provides the converted energy to the power source for recharging.

### CROSS-REFERENCE TO RELATED APPLICATIONS

This application claims priority to U.S. Provisional Patent Application No. 61/075,945, filed on Jun. 26, 2008, which is incorporated herein for all purposes.

### TECHNICAL FIELD

This invention relates to a solar cell used in an implantable medical device and, more particularly, to a solar cell for recharging a power source of an implantable medical device.

### BACKGROUND

Implantable medical devices can be passive or active devices. Passive implantable devices tend to be structural devices (e.g., artificial joints, vascular grafts and artificial valves). Active implantable devices require power to replace or augment an organ's function. Examples of active implantable devices are cardiac pacemakers, cardiac defibrillators and neurological stimulators. Power to these devices may be supplied by an external power source or internal batteries integrated into the implanted device.

Reliability is an important factor for an implantable battery since batteries in implantable devices cannot be replaced easily. Batteries in implantable devices are generally hard-wired at the time of manufacture before the device is hermetically sealed. From that point on, the battery is expected to power the device throughout the useful life of the device. In general, the power source of the implantable device often determines the service life of the implantable device. Batteries generally power an implantable device for five to eight years.

Different types of implantable devices may have different power requirements. Devices with low power consumption can utilize batteries internal to the implantable device. The cardiac pacemaker is one such a device. The cardiac pacemaker uses its battery power for cardiac stimulation, monitoring and data logging, which do not require high power. A one amp-hour battery built using lithium iodine technology provides about five years of operation. The implantable cardiac defibrillator is another such a device. Although a cardiac defibrillator requires a relatively large amount of power when activated, unlike the pacemaker, it does not need to be activated on a continuous basis.

A battery for powering an implantable medical device may include rechargeable cells that use radio frequency transmission to be recharged. This requires an external radio frequency charging device. Potential hazards associated with the use of radio frequency charging include: 1) tissue damage due to radio frequencies; 2) internal implanted device damage due to power, voltage and current transients; 3) malfunction of the external radio frequency charging device causing radio frequency burn or long term tissue damage; and 4) electrocution of the patient using the external charging device due to electrical insulation malfunction.

Therefore, it is desirable to provide a medical device with a power source that does not need to be replaced and that may be easily and safely recharged.

### SUMMARY

The present invention provides a solar cell for an implantable medical device. The solar cell is configured to provide energy to recharge a power source such as a battery. The power source is coupled to a control circuit of the medical device and provides power to the control circuit. The solar cell may be coupled to the power source via a wire and may be distanced from a housing of the medical device. The solar cell may also be attached to the housing or may be disposed in the housing. The medical device may be implanted in the body of a host such that a surface of the solar cell is provided under a layer of skin of the host. The translucent property of skin allows the solar cell to receive light or infrared radiation from outside the body. The solar cell converts the received energy and provides the converted energy to the power source for recharging.

In one embodiment, an implantable medical device is implanted in a body of a host. The implantable medical device includes a housing, a control circuit, a power source and a solar cell. The control circuit is configured to perform one or more functions, and is enclosed within the housing. The power source is coupled to the control circuit and is configured to provide power to the control circuit. The power source is also enclosed within the housing. The solar cell is coupled to the power source and a surface of the solar cell being provided under a layer of skin of the host. The solar cell is configured to receive energy from outside the host, convert the energy for use by the power source and to provide the converted energy to the power source.

In another embodiment, a method for providing energy to a power source of an implantable medical device uses a solar cell. The implantable medical device is implanted in a body of a host. The method includes receiving energy at the solar cell. A surface of the solar cell is provided under a layer of skin of the host. The energy is received from outside the host. The received energy is converted for use with a power source. The converted energy is provided to the power source for recharging the power source.

A further understanding of the nature and advantages of the present invention may be realized by reference to the remaining portions of the specification and the drawings.

### BRIEF DESCRIPTION OF THE DRAWINGS

FIG. 1 is a block diagram of an implantable medical device according to one embodiment of the invention.

FIG. 2 illustrates a perspective view of an implantable medical device with a solar cell attached to a housing thereof, according to one embodiment of the invention.

FIGS. 3 and 4 illustrate side views of a medical device that is implanted within a body of a host.

FIG. 5 illustrates a perspective view of an implantable medical device including solar cells according to one embodiment of the invention.

FIG. 6 illustrates a side view of the medical device shown in FIG. 5 that is implanted within a body of a host.

FIG. 7 illustrates a perspective view of an implantable medical device including solar cells according to one embodiment of the invention.

FIG. 8 is a flow diagram illustrating a method for providing energy to a power source of an implantable medical device using a solar cell according to one embodiment of the invention.

### DETAILED DESCRIPTION

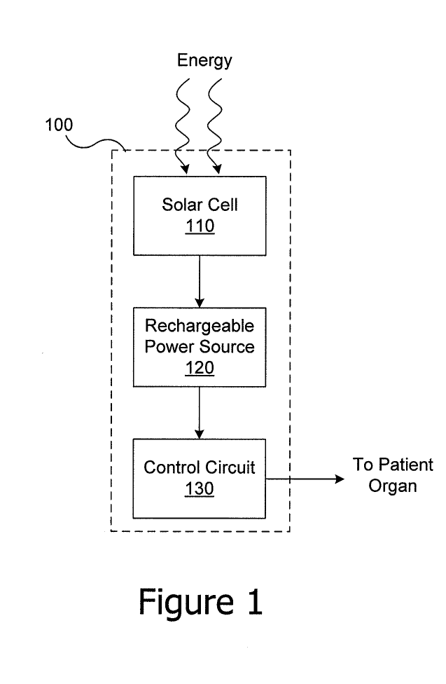

FIG. 1 is a block diagram of an implantable medical device 100 according to one embodiment of the invention. The implantable medical device 100 includes a solar cell 110, a rechargeable power source 120 and a control circuit 130. The medical device 100 is implanted in a body of a host. The solar cell 110 receives energy from a source outside of the body of the host, and converts the received energy into energy for recharging the power source 120. For example, the solar cell 110 may receive photons from light through a layer of skin of the host and convert the photons to electrical current or voltage, as is known in the art. The rechargeable power source 120 is then used to power the control circuit 130 which controls the function of the implantable medical device 100. For example, the implantable medical device 100 may be a cardiac defibrillator and the control circuit 130 may be configured to discharge electrical energy to a patient's heart to restore a normal rhythmic heartbeat.

The solar cell 110 may be optimized to receive energy in a wavelength range of between about 300 nm and 900 nm. In one embodiment, as described above, the solar cell 110 receives photons (e.g. in the form of sunlight or artificial light) as an energy source. In another embodiment, the solar cell 110 is optimized for electromagnetic radiation. Near infrared radiation is electromagnetic radiation of a wavelength longer than that of visible light, but shorter than that of radio waves. Infrared radiation has wavelengths between about 750 nm and 1 mm. In one embodiment, the solar cell 110 is optimized for a wavelength of approximately 880 nm to receive energy through translucent skin, body tissue and clothing. In another embodiment, the solar cell 110 is optimized for visible light for which skin is translucent (i.e., a wavelength range of between about 380 nm and 750 nm). A wavelength of less than 380 nm range is for capturing ultraviolet light (i.e., a portion of light created by the sun).

In one embodiment, the solar cell 110 is a monolithic photovoltaic string of solar cells to provide 4V to 12V to recharge the power source 120 of the implantable medical device 100. In another embodiment, the solar cell 110 is a single crystalline solar cell. The single crystalline solar cell may require a fly back converter or a voltage charge pump circuit to operate effectively.

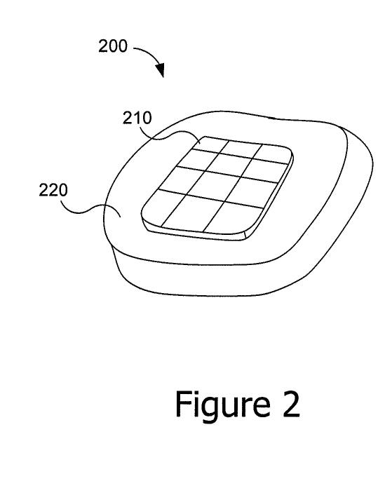

FIG. 2 illustrates a perspective view of an implantable medical device 200 with a solar cell 210 attached to a housing 220 thereof, according to one embodiment of the invention. The implantable medical device 200 may be any active device that is operable within a body of a host (i.e., a patient). For example, the medical device 200 may be a cardiac defibrillator or a pacemaker. The solar cell 210 is attached to the housing 220 of the implantable medical device 200. As discussed above, the solar cell 220 is provided to receive energy from an outside source, convert the received energy to a voltage or an electrical current, and provide the voltage/current to the power source in the implantable medical device 200 for recharging the power source. The control circuit for operating the medical device 200 and the power source for providing power to the control circuit are enclosed within the housing 220 of the medical device 200. In one embodiment, the solar cell 210 has a dimension that is smaller than a dimension of the housing 220. For example, the housing 220 may have a dimension of approximately 10 mm² to approximately 5000 mm² and the solar cell 210 has a dimension of approximately 3 mm² to approximately 1000 mm².

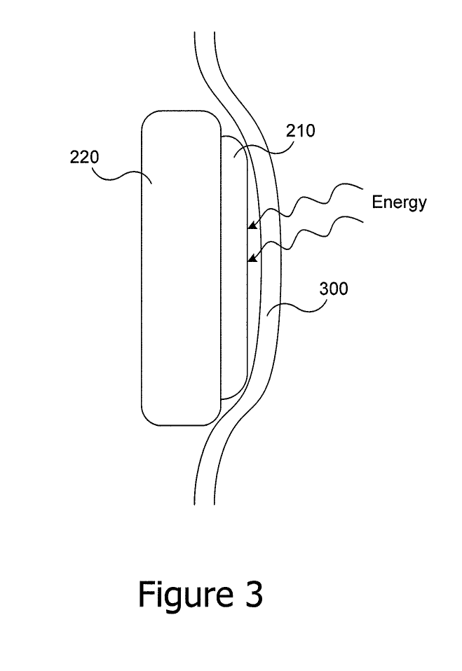

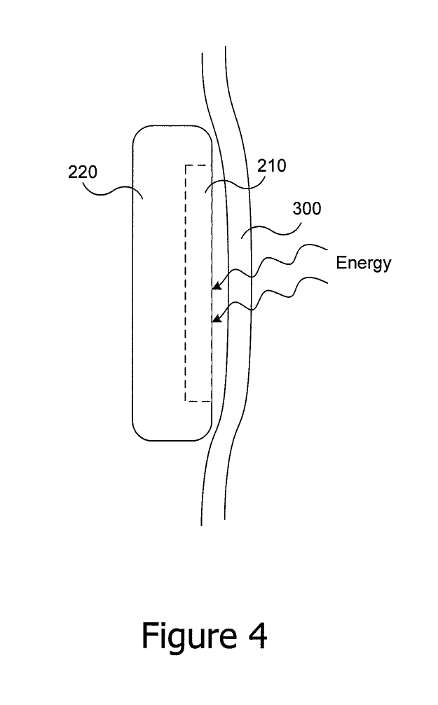

FIGS. 3 and 4 illustrate side views of the medical device that is implanted within a body of a host. The medical device is implanted such that the solar cell 210 may be provided between the housing 220 and under a layer of skin 300 of the host. As shown in FIG. 3, the solar cell 210 is mounted on the housing 220 of the medical device. An outer surface of the solar cell 210 is positioned under the layer of skin 300 such that energy may be received at the solar cell from outside of the host. As illustrated in FIG. 4, the solar cell 210 may be provided within the housing 220 such that an outer surface of the solar cell 210 is flush with an outer surface of the housing 220. Similar to the medical device illustrated in FIG. 3, the outer surface of the solar cell 210 shown in FIG. 4 is positioned under the layer of skin 300 such that energy may be received at the solar cell from outside of the host. In one embodiment, the solar cell 210 is disposed under the layer of skin 300 at a depth of between one and ten millimeters.

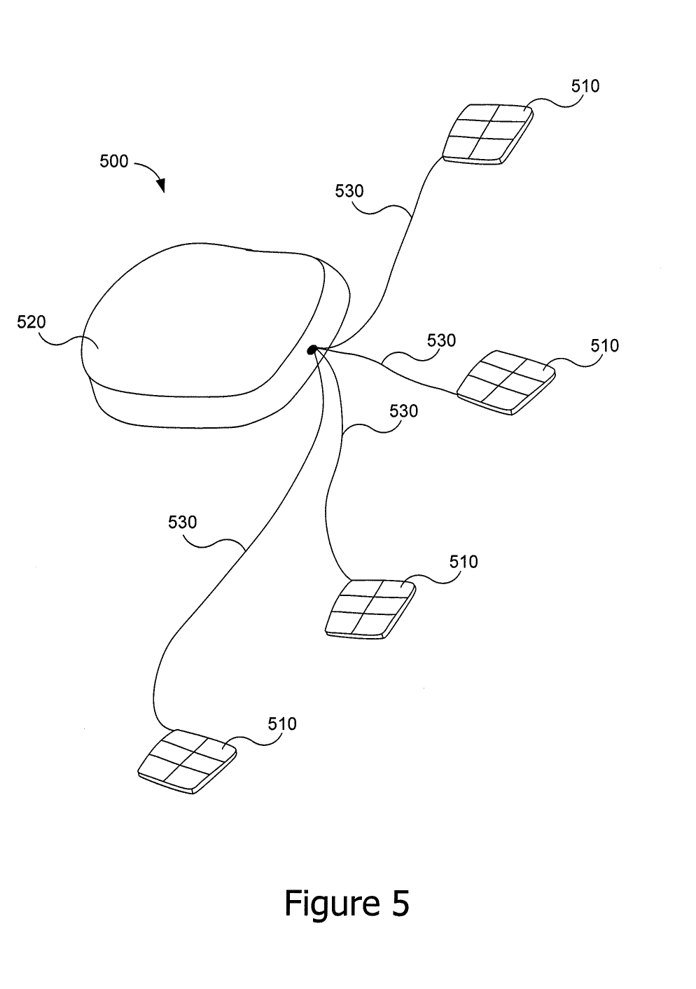

FIG. 5 illustrates a perspective view of an implantable medical device 500 including solar cells 510 according to one embodiment of the invention. The solar cells 510 are not directly attached to a housing 520 of the medical device 500. Rather, flexible wires 530 are provided to couple the solar cells 510 to the medical device 500. In one embodiment, each solar cell 510 has a dimension of approximately 3 mm² to approximately 1000 mm².

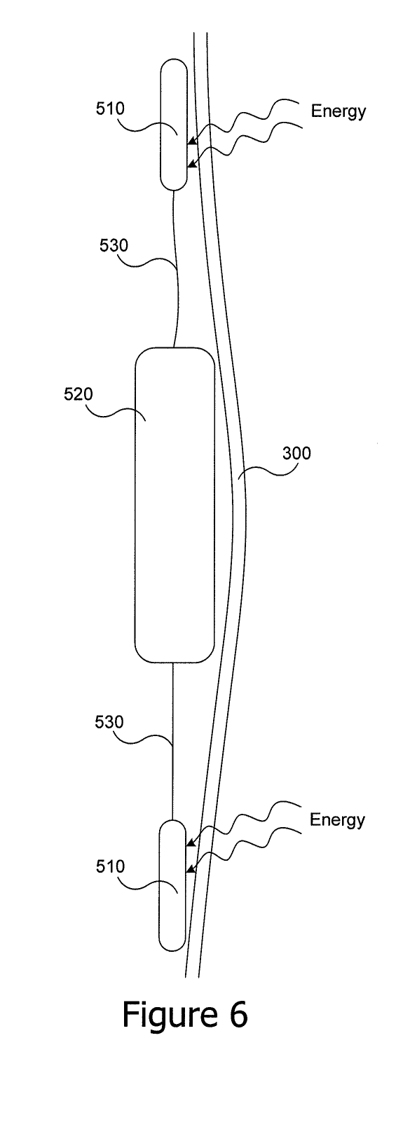

The embodiment illustrated in FIG. 5 allows the solar cells 510 to be positioned in the body of the host at a location that receives more energy for recharging the power source of the implantable medical device 500. For example, as shown in FIG. 6, the solar cells 510 may be positioned under the skin 300 of a person requiring such a medical device and distanced away from the housing 520 of the medical device 500 in an area of the body that may be more accessible to receiving energy (e.g., under a layer of skin that is not covered with clothing).

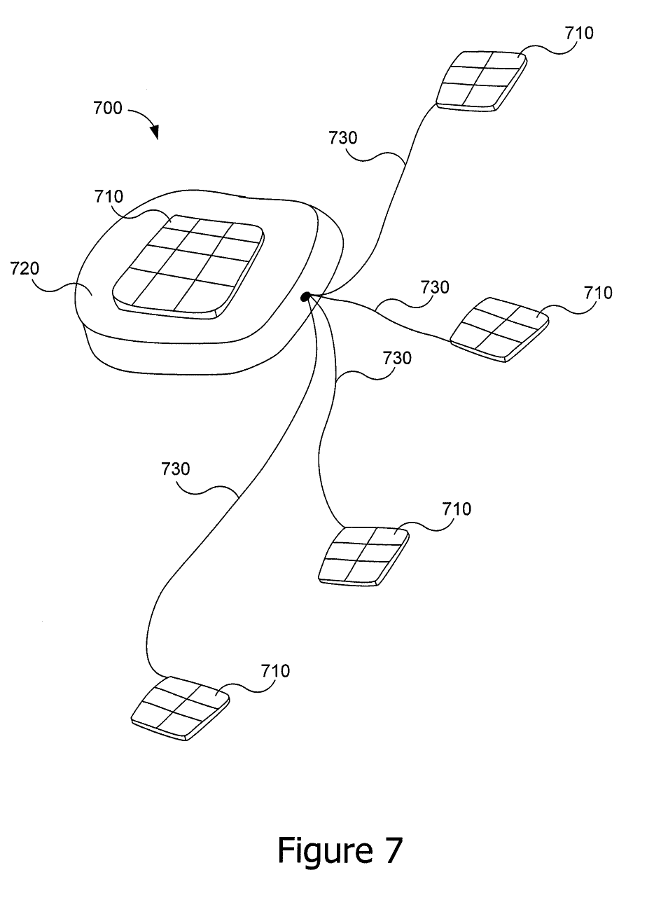

FIG. 7 illustrates an implantable medical device 700 including solar cells 710 according to one embodiment of the invention. In this embodiment, a solar cell 710 is provided on a housing 720 of the medical device 700, as described with reference to FIGS. 2-4. Additional solar cells 710 are coupled to the medical device 700 via flexible wires 730, as described with reference to FIGS. 5 and 6.

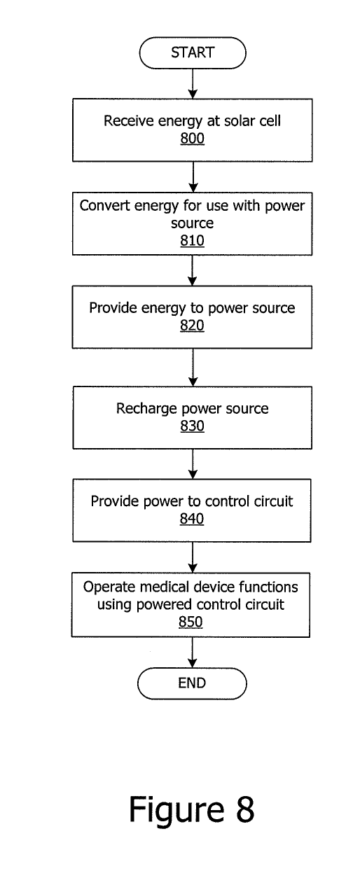

FIG. 8 is a flow diagram illustrating a method for providing energy to a power source of an implantable medical device using a solar cell. The implantable medical device may be any active medical device that is operable within a body of a host. For example, the medical device may be a cardiac defibrillator or a pacemaker.

Energy is received at the solar cell (step 800). A surface of the solar cell is provided under a layer of skin of the host patient. In one embodiment, the solar cell is disposed under the layer of skin at a depth of between one and ten millimeters. The energy is received from outside of the host. The solar cell uses the translucent property of skin, body issue or clothing to receive the energy. In one implementation, the solar cell is optimized for a wavelength to receive energy through translucent skin, body tissue and/or clothing. The solar cell may be optimized for a range of wavelengths between 300 nm and 900 nm. The received energy may be light or infrared radiation. The solar cell may be a monolithic photovoltaic string of solar cells or a single crystalline solar cell.

The received energy is converted for use with a power source (step 810). The energy may be converted by the solar cell to voltage or electrical current. The power source may be any electrical component used for providing power to a circuit. For example, the power source may be a battery or a capacitor.

The converted energy is provided to the power source (step 820). The converted energy may be provided to the power source via a wire. The wire allows the solar cell to be displaced from the power source such that the solar cell may be positioned in the body of the host at an area that is more likely to receive energy from outside the host (e.g., under skin that is not covered with clothing).

The converted energy is used to recharge the power source (step 830). The recharged power source provide power to a control circuit of the implantable medical device (step 840). The powered control circuit is used to operate functions of the implantable medical device (step 850). For example, the control circuit may be used to restore a normal rhythmic heartbeat of the host. Processing then terminates.

As disclosed above, an implantable medical device includes a solar cell configured to provide energy to recharge a power source such as a battery or a capacitor. The power source is coupled to a control circuit of the medical device and provides power to the control circuit. The solar cell may be coupled to the power source via a wire and may be distanced from a housing of the medical device. The solar cell may also be attached to the housing or may be disposed in the housing. The medical device may be implanted in the body of a host such that a surface of the solar cell is provided under a layer of skin of the host. The translucent property of skin allows the solar cell to receive light or infrared radiation from outside the body. The solar cell converts the received energy to an electrical current and provides the current to the power source for recharging the power source.

While the invention has been particularly shown and described with reference to specific embodiments, it will be understood by those skilled in the art that the foregoing and other changes in the form and details may be made therein without departing from the spirit or scope of the invention. Therefore, the scope of this invention should not be limited to the embodiments described above, and should instead be defined by the following claims.

What is claimed is:

1. An implantable medical device being implanted in a body of a host, the implantable medical device comprising:
a housing;
a control circuit configured to perform one or more functions, wherein the control circuit is enclosed within the housing;
a power source coupled to the control circuit and configured to provide power to the control circuit, wherein the power source is enclosed within the housing; and
a solar cell coupled to the power source, a surface of the solar cell being provided under a layer of skin of the host, wherein the solar cell is configured to receive energy from outside the host, convert the energy for use by the power source and to provide the converted energy to the power source.

2. The device of claim 1, wherein the solar cell is coupled to the power source via a wire.

3. The device of claim 1, wherein the solar cell is attached to the housing.

4. The device of claim 1, wherein the solar cell is disposed in the housing, a surface of the solar cell being flush with a surface of the housing

5. The device of claim 1, wherein the power source is a battery.

6. The device of claim 1, wherein the power source is a capacitor.

7. The device of claim 1, wherein the implantable medical device is a cardiac defibrillator or a pacemaker.

8. The device of claim 1, wherein the converted energy provided by the solar cell is a voltage or an electrical current that recharges the power source.

9. The device of claim 1, wherein the solar cell is configured to receive light and convert the received light to the energy provided to the power source.

10. The device of claim 1, wherein the solar cell is configured to receive infrared radiation and convert the received infrared radiation to the energy provided to the power source.

11. The device of claim 1, wherein the solar cell is a monolithic photovoltaic string of solar cells.

12. The device of claim 1, wherein the solar cell is a single crystalline solar cell.

13. The device of claim 1, wherein the solar cell is optimized for a wavelength between 300 nm and 900 nm.

14. A method for providing energy to a power source of an implantable medical device using a solar cell, the implantable medical device being implanted in a body of a host, the method comprising:
receiving energy at the solar cell, wherein a surface of the solar cell is provided under a layer of skin of the host, the energy being received from outside the host;
converting the received energy for use with a power source;
providing the converted energy to the power source; and
recharging the power source.

15. The method of claim 14, further comprising:
providing power from the power source to a control circuit, wherein the control circuit is configured to perform one or more functions of the implantable medical device.

16. The method of claim 14, wherein the converted energy is provided to the power source via a wire.

17. A system for providing energy to a power source of an implantable medical device, the implantable medical device being implanted in a body of a host, the method comprising:
means for receiving energy having a surface provided under a layer of skin of the host, wherein the energy is received from outside the host;
means for converting the received energy for use with a power source;
means for providing the converted energy to the power source;
means for recharging the power source; and
means for providing power from the power source to a control circuit, wherein the control circuit is configured to perform one or more functions of the implantable medical device.

18. A method for performing a medical procedure, the method comprising:
implanting a medical device in a body of a host under a layer of skin of the host, wherein the medical device comprises:
a housing;
a control circuit configured to perform one or more functions, wherein the control circuit is enclosed within the housing;
a power source coupled to the control circuit and configured to provide power to the control circuit, wherein the power source is enclosed within the housing; and
a solar cell coupled to the power source, a surface of the solar cell being provided under the layer of skin of the host, wherein the solar cell is configured to receive energy from outside the host, convert the energy for use by the power source and to provide the converted energy to the power source.
